(19) United States
(12) Patent Application Publication
(10) Pub. No.: US 2008/0069830 A1
(43) Pub. Date: Mar. 20, 2008

(54) Dna Sequences, Peptides, Antibodies and Vaccines for Prevention and Treatment of Sars

(76) Inventors: Stephen L. Hoffman, Gaithersburg, MD (US); Kim Lee Sim, Gaithersburg, MD (US); Hong Liang, Shoreline, WA (US)

Correspondence Address: DAVID S. DOLBERG, 2163 MEEKER AVENUE, SUITE# 116, RICHMOND, CA, 94804, US

(21) Appl. No.: 10/562,164

(22) PCT Filed: Jun. 24, 2004
(86) PCT No.: PCT/US04/20068

§ 371 Date: Jun. 19, 2006

Related U.S. Application Data
(60) Provisional application No. 60/480,953, filed on Jun. 24, 2003.

(51) Int. Cl. A61K 39/00; A61K 31/70; A61P 43/00; C07H 21/00; C07K 14/00; C07K 16/18

(52) U.S. Cl. 424/186.1; 514/44; 530/350; 530/387.9; 530/388.1; 536/23.72

(57) ABSTRACT

The invention comprises novel DNA, protein, and peptide sequences and novel vaccines, therapeutics and antibodies created with the novel DNA and peptides to prevent or treat SARS by administration of recombinant proteins, synthetic peptides, recombinant viruses, recombinant bacteria, DNA plasmids, RNA plasmids, or other molecular constructs or delivery systems.

### REFERENCE TO RELATED PATENT APPLICATIONS

This application claims priority from U.S. Provisional Application No. 60/480,953, filed 24 Jun. 2003, incorporated herein by reference in its entirety.

### FIELD OF THE INVENTION

The invention relates to the field of vaccines, therapeutics and antibodies. More particularly, the inventions pertains to DNA, protein, and peptide sequences used to construct vaccines, therapeutics, and antibodies for the prevention and/or treatment of SARS; the vaccines, therapeutics and antibodies created and produced using these novel DNA, protein, or peptide sequences; and a method of treatment and/or prevention of SARS using these vaccines, therapeutics and antibodies for the prevention and/or treatment created and produced using these novel DNA, protein, or peptide sequences.

### BACKGROUND OF THE INVENTION

Severe acute respiratory syndrome (SARS) has recently been reported in Asia, North America, and Europe (Rota, P A, Oberste, M S, Monroe, S S et. al. Characterization of a Novel Coronavirus Associated with Severe Acute Respiratory Syndrome 2003 Science 300:1394-1399; Ksiazek, T G, Erdman, D, Goldsmith, C S, et al. A novel coronavirus associated with severe acute respiratory syndrome. 2003 N Engl J Med 348:1953-66; Comments on the reported isolation of viruses related to the SARS coronavirus in wild animals in southern China—available at http://www.who.int/csr/don/2003 05 23b/en/; www.cdc.gov/ncidod/sars/ and www.who.int/csr/sars/en/). Data and information on SARS is updated frequently by the CDC. An update with references of Jun. 4, 2003 by the CDC to the president of the Infectious Disease Society of America was disseminated to the membership of the IDSA and provided most of the background information below.

In March 2003, a novel coronavirus (SARS-CoV) was discovered in association with cases of severe acute respiratory syndrome (SARS). Phylogenetic analyses and sequence comparisons showed that SARS-CoV is not closely related to any of the previously characterized coronaviruses. The WHO has reported that research teams in Hong Kong and Shenzhen, China detected several coronaviruses closely related genetically to the SARS-CoV in two animal species taken from a market (masked palm civet and raccoon-dog). The study also found that one additional species (Chinese ferret badger) elicited antibodies against the SARS coronavirus. Sequencing of viruses isolated from these animals demonstrated that, with the exception of a small additional sequence, the viruses are identical with the human SARS virus. Much more research is needed before any firm conclusions can be reached on the potential role of animals in the transmission of SARS.

During Nov. 1, 2002-May 28, 2003, a total of 8,240 SARS cases were reported to WHO from 28 countries, including the United States; 745 deaths (case-fatality proportion: 9.0%) have been reported. 363 SARS cases have been identified in the United States. There have been reports from 41 states and Puerto Rico, with 297(82%) cases classified as suspect SARS and 66 (18%) classified as probable SARS (more severe illnesses characterized by the presence of pneumonia or acute respiratory distress syndrome) (MMWR 2003;52:500-501). No SARS-related deaths have been reported in the United States. (World Health Organization. Cumulative number of reported cases of severe acute respiratory syndrome (SARS). http://www.who.int/csr/sarscountry/2003 05 28/en.; CDC. Updated interim U.S. case definition of severe acute respiratory syndrome (SARS). http://www.cdc.gov/ncidod/sars/casedefmition.htm; CDC. Severe acute respiratory syndrome (SARS) and coronavirus testing—United States, 2003. 2003 MMWR 52:297-302 http://www.cdc.gov/mmwr/preview/mmwrhtml/mm5214a1.htm; CDC. Update: severe acute respiratory syndrome—United States, 2003. 2003MMWR52:357-60. http://www.cdc. gov/mmwr/preview/mmwrhtml/mm5216a1.htm;

Peiris, J S M, Chu, C M, Cheng, V C C et. al. Clinical progression and viral load in a community outbreak of coronavirus-associated SARS pneumonia: a prospective study. 2003 Lancet 361 1767; CDC. Severe Acute Respiratory Syndrome—Singapore, 2003. MMWR 2003:52;405-411; CDC. Cluster of Severe Acute Respiratory Syndrome Cases Among Protected Health-Care Workers—Toronto, Canada, April 2003. 2003 MMWR 52;433-436.

Worldwide, a significant proportion of transmissions have occurred within the healthcare setting, and most transmissions originated from patients for whom infection control precautions had not yet been applied. For most patients the incubation period falls between 4-6 days. To date there has been no evidence of transmission from asymptomatic patients, although transmission may occur early in the course of illness.

Communicability appears to peak in the second week of illness, and viral excretion may peak during this time as well. The duration of communicability following onset of symptoms is unknown. Evidence of SARS-CoV RNA has been detected by RT-PCR in respiratory secretions, urine, and stool for several weeks following symptom onset, although it is unclear whether detection of viral RNA represents the presence of viable virus capable of causing infection. The efficiency of transmission appears to be highly variable between patients. Many cases have been associated with little or no secondary transmission, but a small minority of patients appears to have accounted for many secondary cases. The factors associated with high rates of secondary transmission are poorly understood. In most cases transmission appears to require close contact, most likely via large respiratory droplets or direct contact. Several investigators have reported that SARS-CoV may remain viable on environmental surfaces for 1-2 days, raising the possibility of fomite transmission, but these observations require confirmation. Airborne transmission, although not apparently a major mode of transmission, may have played a role in some reported clusters of infection, particularly during the performance of aerosol generating medical procedures.

Pathogenesis: Although the pathogenesis of SARS remains poorly understood, some investigators have hypothesized that the disease process may be, in part, immune mediated, raising the hypothesis that immunomodulatory agents such as corticosteroids may have a role in treatment. No controlled studies have been conducted and the efficacy of this approach is unknown. Some investigators have hypothesized that SARS-CoV-specific immune globulin may be of benefit. However, in some animal coronavirus infections antibody responses may produce an antibody-dependant enhancement of disease.

Diagnostic Tests: At this time, tests for SARS-CoV are still being refined, and the sensitivity and specificity are uncertain and still being evaluated. The majority are assays that measure antibodies to the virus. Reverse transcription-polymerase chain reaction (RT-PCR) testing is also available. This test can detect SARS-CoV RNA in clinical specimens, including serum, stool, and nasal secretions. Viral isolation for SARS-CoV also has been done. In these studies, clinical specimens from SARS patients are co-cultured with well-characterized cell lines, and then laboratorians look for evidence of SARS-CoV replication in these cultured cells.

Therapy: All current therapy is supportive. At this time there are no specific therapeutic regimens with proven efficacy. Ribavirin appears to lack in vitro activity against SARS-CoV. Alpha and beta interferons may have some in vitro activity against the SARS-CoV, but the endogenous cytokine response in SARS is poorly understood and it remains unclear whether administration of exogenous interferons might be beneficial. Some 3C proteinase inhibitors have shown promising in vitro activity, but there are no licensed 3C proteinase inhibitors currently available for human use.

Prevention: All current prevention is by identification and isolation of cases. There is no vaccine available. Controlled therapeutic trials are needed.

Rationale for the Invention: A vaccine would be the ideal intervention for preventing SARS. However, there is no vaccine for SARS, and no consensus on the best approach to SARS vaccine development. However, there has already been considerable discussion regarding different approaches to SARS vaccine development, which was summarized at a May 30, 2003 meeting at the National Institutes of Health (http://www.niaid.nih.gov/sars meeting.htm).

It has been demonstrated that antibody interference with virion attachment to target cells is a major neutralization mechanism. (Matsumi, S, et al. Neutralizing monoclonal antibody against an external envelope glycoprotein (gp110) of SIV mac251. 1995 AIDS Res Hum Retroviruses (11) 501-8; Samuelsson, A. et al. Chimeric macaque/human Fab molecules neutralize simian immunodeficiency virus. 1995 Virology (207) 495-502; Sattentau, Q J, et al. Antibody neutralization of HIV-1 and the potential for vaccine design. 1999 Immunol. Lett. (66) 143-9; Wobus, C E, et al. Monoclonal antibodies against the adeno-associated virus type 2 (AAV-2) capsid: epitope mapping and identification of capsid domains involved in AAV-2-cell interaction and neutralization of AAV-2 infection. 2000 J. Virol. (74)9281-93). Evidence supporting a role for the Coronavirus spike protein projections as agents of organ tropism and pathogenesis has been reviewed. (Gallagher, T M and Buchmeier; Coronavirus spike proteins in viral entry and pathogenesis 2001 Virology 279:371-374).

### SUMMARY OF THE INVENTION

Applicants have discovered and created novel gene sequences based on the Spike protein, also called S or E2 glycoprotein, of SARS CoV that, when used to express the polypeptide in a DNA plasmid, recombinant virus, recombinant bacteria, replicon, or other DNA or RNA based vaccine delivery system, or to produce a recombinant protein or synthetic peptide, can enable the creation and production of vaccines, therapeutics, and or antibodies that are useful to treat and/or prevent SARS. In the context of the present invention, a “replicon” is a an autonomously replicating piece of DNA other than the chromosome, such as a plasmid or a phage.

More particularly, applicants have discovered and created novel gene, novel protein, and novel peptide sequences that can be used to produce vaccines, therapeutics, and/or antibodies that will provide effective treatment or prevention of SARS by their administration on the skin, in the skin, in the subcutaneous tissue, in muscles, in the blood stream, or on any mucosal surface, or by oral ingestion.

Furthermore, applicants have discovered and created novel gene, protein, and peptide sequences that can be used to produce vaccines, therapeutics, and/or antibodies that will provide effective treatment or prevention of SARS by their administration on the skin, in the skin, in the subcutaneous tissue, in muscles, in the blood stream, or on any mucosal surface, or by oral ingestion, and therefore the method of treating and preventing SARS.

Accordingly, it is an object of this invention to provide a family of Region I Spike Protein SARS CoV Native no glycosylation (RISPSCoVNNG) polypeptide vaccines that protects a mammal, a human, or an individual or group of individuals against Severe Acute Respiratory Syndrome (SARS).

It is an object of this invention to provide a family of RISPSCoVNNG polypeptide vaccines that attenuate the effects of SARS in a mammal, a human, or an individual or group of individuals.

It is an object of this invention to provide DNA vaccines, DNA plasmid vaccines, replicon vaccines, recombinant viral, bacterial and parasitic vaccines encoding the RISPSCoVNNG polypeptide or sub regions thereof that protect a mammal, a human, or an individual or group of individuals against Severe Acute Respiratory Syndrome (SARS).

It is an object of this invention to provide DNA vaccines replicon vaccines, recombinant viral, bacterial and parasitic vaccines encoding the RISPSCoVNNG polypeptide or sub regions thereof that attenuate the effects of SARS in a mammal, a human, or an individual or group of individuals.

It is a further object of this invention to provide polypeptides from the Region 1 of the SARS CoV Spike protein in which one or more of the potential N-linked glycosylation sites have had a peptide residue substituted to prevent glycosylation.

It is a further object of this invention to provide polypeptides from the Region 1 of the SARS CoV Spike protein in which all potential N-linked glycosylation sites have had a peptide residue substituted to prevent glycosylation.

It is an additional object of this invention to provide a DNA molecule with a nucleic acid sequence encoding Region I of the SARS CoV Spike protein or sub regions thereof in which one or more of the potential N-linked glycosylation sites have had a peptide residue substituted to prevent glycosylation.

It is an additional object of this invention to provide a DNA molecule with a nucleic acid sequence encoding Region I of the SARS CoV Spike protein or sub regions thereof in which all of the potential N-linked glycosylation sites have had a peptide residue substituted to prevent glycosylation.

It is yet another object of this invention to provide a DNA molecule with a nucleic acid sequence encoding Region I of the SARS CoV Spike protein or sub regions thereof in which one or more of the potential N-linked glycosylation sites have had a peptide residue substituted to prevent glycosylation and, taking advantage of the degeneracy of the DNA code, primary sequence has been altered to optimize expression and enhance immunogenicity for antibodies and thus, protective efficacy.

It is yet another object of this invention to provide a DNA molecule with a nucleic acid sequence encoding Region I of the SARS CoV Spike protein or sub regions thereof in which all of the potential N-linked glycosylation sites have had a peptide residue substituted to prevent glycosylation and, taking advantage of the degeneracy of the DNA code, the primary sequence has been altered to optimize expression and enhance immunogenicity for antibodies and thus, protective efficacy.

It is also an object of this invention to provide polyclonal and monoclonal antibodies that recognize, and neutralize polypeptides from the Region I of the SARS CoV Spike protein in which one or more of the potential N-linked glycosylation sites have had a peptide residue substituted to prevent glycosylation.

It is also an object of this invention to provide polyclonal and monoclonal antibodies that recognize, and neutralize polypeptides from the Region 1 of the SARS CoV Spike protein in which all potential N-linked glycosylation sites have had a peptide residue substituted to prevent glycosylation.

It is a further object of this invention to provide polypeptides from the Region 1 of the SARS CoV Spike protein in which all potential N-linked glycosylation sites have been changed to Alanine.

It is an additional object of this invention to provide a DNA molecule with a nucleic acid sequence encoding Region I of the SARS CoV Spike protein or sub regions thereof in which one or more of the potential N-linked glycosylation sites have been changed to Alanine.

It is an additional object of this invention to provide a DNA molecule with a nucleic acid sequence encoding Region I of the SARS CoV Spike protein or sub regions thereof in which all of the potential N-linked glycosylation sites have been changed to Alanine.

It is yet another object of this invention to provide a DNA molecule with a nucleic acid sequence encoding Region I of the SARS CoV Spike protein or sub regions thereof in which one or more of the potential N-linked glycosylation sites have been changed to Alanine and, taking advantage of the degeneracy of the DNA code, primary sequence has been altered to optimize expression and enhance immunogenicity for antibodies and thus, protective efficacy.

It is yet another object of this invention to provide a DNA molecule with a nucleic acid sequence encoding Region I of the SARS CoV Spike protein or sub regions thereof in which all of the potential N-linked glycosylation sites have been changed to Alanine and, taking advantage of the degeneracy of the DNA code, the primary sequence has been altered to optimize expression and enhance immunogenicity for antibodies and thus, protective efficacy.

It is also an object of this invention to provide polyclonal and monoclonal antibodies that recognize, and neutralize polypeptides from the Region I of the SARS CoV Spike protein in which one or more of the potential N-linked glycosylation sites have been changed to Alanine.

It is also an object of this invention to provide polyclonal and monoclonal antibodies that recognize, and neutralize polypeptides from the Region 1 of the SARS CoV Spike protein in which all potential N-linked glycosylation sites have been changed to Alanine.

In consideration of the foregoing objects of the invention and others which will become apparent from the context of the disclosure the Applicants hereby provide the following:
Novel DNA molecules comprising a nucleotide sequence encoding various sub regions of Region I Spike Protein SARS CoV native no glycosylation (R1SPSCoVNNG).
Novel Polypeptides of various sub regions of RISPSCoVNNG in which one or more potential N-linked glycosylation sites are substituted with Alanine.
Vaccines for the prevention, attenuation or treatment of Severe Acute Respiratory Syndrome (SARS) in mammals comprising a DNA molecule that encodes one or more sub regions of R1SPSCoVNNG.
Vaccines for the prevention, attenuation or treatment of Severe Acute Respiratory Syndrome (SARS) in mammals comprising a sub region of RIIPSCoVNNG
Antibodies against the novel peptides from RISPSCoVNNG

### DESCRIPTION OF THE DRAWINGS

FIG. 1—Depiction of structure of a typical Coronavirus such as SARS CoV. The structure labeled “S” is the Spike Protein, which is also called the E2 glycoprotein; other features of the viral structure are indicated (H-protein, HE-protein, and RNA genome).

FIG. 2—Depiction of a host cell interaction with a typical Coronavirus such as SARS CoV.

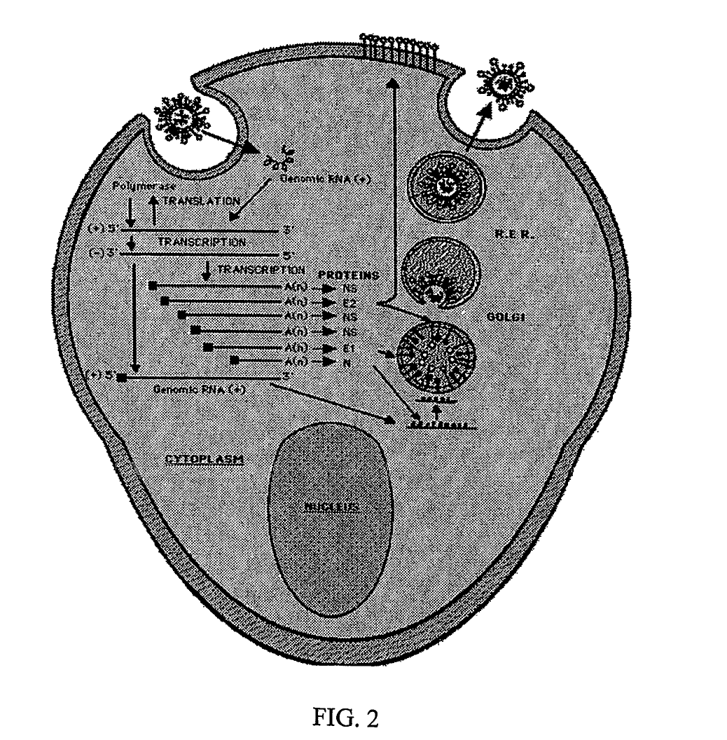

The virus enters cells via endocytosis and membrane fusion. This critical step in the pathogenesis of the infection and disease is thought to probably be mediated by the E2 glycoprotein, which is represented by the small circles on the surface of the virus.

FIG. 3—Depiction of the genomic RNA of SARS CoV.

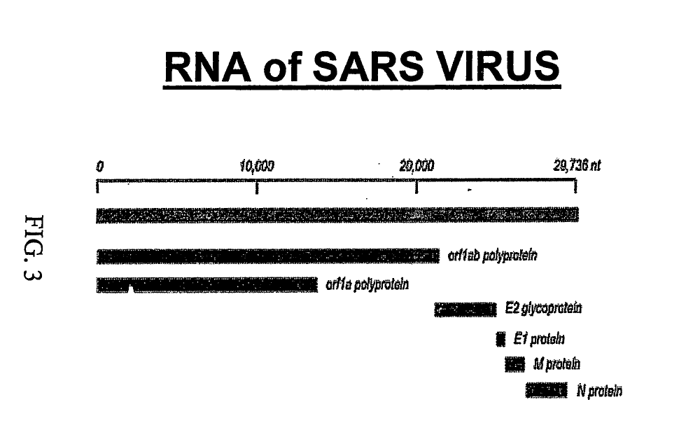

The SARS CoV is a non-segmented, single-stranded, (+) sense RNA, which was 29.736 kb in size in one of the first sequenced isolates. This makes it the longest of any RNA virus currently known. The genome has a 5′ methylated cap and 3′ poly-A and functions directly as mRNA. The RNA encoding the E2 glycoprotein (also known as the S or Spike protein) is 3.768 kb.

FIG. 4—Schematic of spike protein fragments (Regions I-IV) that are targeted for expression and DNA plasmid construction, wherein “*” denotes glycosylation sites.

FIG. 5—Schematic representation of SARS Spike protein Pichia pastoris expression constructs depicting portions of the N-terminal and C-terminal sequences of each polypeptide fragment, reiterated in the Table below:

| Construct | N-terminus | C-terminus |
|---|---|---|
| pPICZαA/SPv5/18 | AVDCS | VFNG |
| pPICZαA/SPv8/11 | VLY | GYHL |
| pPICZαA/SPv6/33 | VLY | YQDV |
| pPICZαA/SP/v7/35 | AEQDRN | GYHL |

FIG. 6—SARS expression construct verification by restriction digestion. Ethidium bromide-stained agarose gel shows gene fragments verified by Xhol/XbaI restriction mapping.

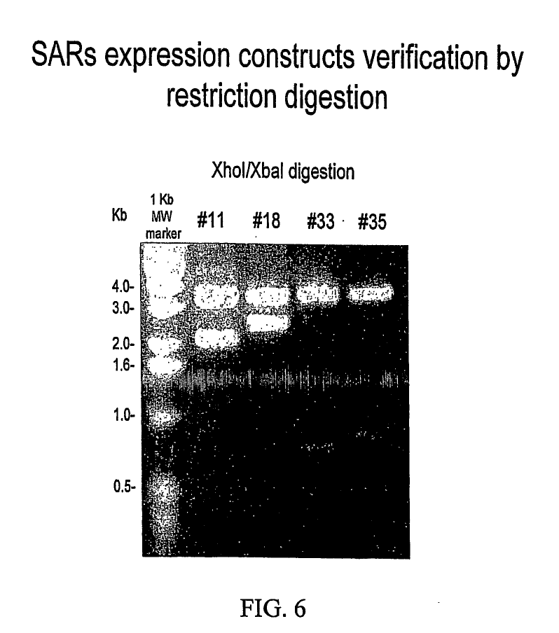

FIG. 7—ELISA titers of rabbits immunized with small peptides from Region I of the native sequence. Rabbits 47299 and 47300 were immunized with 01-11-14-1-pro; rabbits 47297 and 47298 were immunized with 03-11-14-2-pro, as shown in the Table below:

| | | |
|---|---|---|
| Peptide | Amino Acid Sequence | SEQ ID NO. |

| | | |
|---|---|---|
| 03-11-14-1-pro | QILPDPLKPTKRSFI | 27 |
| 03-11-14-2-pro | TRNIDATSTGNYNYKY | 28 |

FIG. 8—Image of a Western blot gel showing that polyclonal rabbit sera raised against the native Region I peptides recognized the Spike glycoprotein expressed in mammalian cells: column 1 was loaded with molecular weight standards (Seeblue Plus); column 2, 20 μl of Complete DMEM media+10% Fetal Bovine Serum control media; column 3, 20 μl of Complete DMEM media+10% Fetal Bovine Serum media from HEK293 cell transfected with Spike protein; column 4, 20 Ml of OptiMEM® control media (GIBCO Laboratories, Inc., Grand Island, N.Y.); column 5, 20 μl of OptiMEM® media; column 6, 1.2 μg of Spike protein produced by VRC; column 7, 1.1 μg of Spike protein produced by PP. Sera from each of the rabbits indicated were incubated with the processed gel portions; detection of bound antibodies was carried out using standard Western blot methods.

### DESCRIPTION OF THE SEQUENCE LISTING AND TABLES

With regard to sequence numbering the convention followed in this application is as follows: all peptide residue numbering corresponds to the amino acid numbering of the native spike protein sequence—SEQ ID NO: 2; all DNA sequence residue numbering corresponds to the DNA base pair numbering of the native spike protein sequence—SEQ ID NO: 1.

SEQ ID NO: 1 is a nucleotide sequence encoding SARS Coronavirus Urbani S-protein (corresponding to the gene AY278741).

SEQ ID NO: 2 is an amino acid sequence for the full length S-protein from accession number AAP13441 (corresponding to the gene AY278741).

SEQ ID NO: 3 is an amino acid sequence for a fragment of wild-type Spike protein identified herein as Region I (aa 275-1081 of SEQ ID NO: 2).

SEQ ID NO: 4 is an amino acid sequence for a modified Spike protein fragment corresponding to aa 275-1081 of SEQ ID NO: 2, having 9 glycosylation sites eliminated.

SEQ ID NO: 5 is a nucleotide sequence encoding Spike protein fragment corresponding to aa 275-1081 of SEQ ID NO: 4, using native virus codons.

SEQ ID NO: 6 is a nucleotide sequence encoding Spike protein fragment corresponding to aa 275-1081 of SEQ ID NO: 4, using altered codons to enhance expression.

SEQ ID NO: 7 is the amino acid sequence shown as SEQ ID NO: 2,with potential glycosylation sites indicated.

SEQ ID NO: 8 is an amino acid sequence of Region II peptide corresponding to amino acids 354-601 as shown in of SEQ ID NO: 4.

SEQ ID NO: 9 is an amino acid sequence of a peptide corresponding to amino acids 754-1031 as shown in SEQ ID NO: 4 (Region III).

SEQ ID NO: 10 is an amino acid sequence of a peptide corresponding to amino acids 354-1031 of SEQ ID NO: 4 (Region IV).

SEQ ID NO: 11 is an amino acid sequence of a peptide corresponding to amino acids 354-601 of SEQ ID NO: 3.

SEQ ID NO: 12 is an amino acid sequence of a peptide corresponding to amino acids 754-1031 of SEQ ID NO: 3.

SEQ ID NO: 13 is an amino acid sequence of a peptide corresponding to amino acids 354-1031 of SEQ ID NO: 3.

SEQ ID NO: 14 is an amino acid sequence of a peptide corresponding to amino acids 275-1031 of SEQ ID NO: 3.

SEQ ID NO: 15 is a linker for nucleotide primer oligo #1.

SEQ ID NO: 16 is a linker for nucleotide primer oligo #2.

SEQ ID NO: 17 is a linker for nucleotide primer oligo #3.

SEQ ID NO: 18 is a linker for nucleotide primer oligo #4.

SEQ ID NO: 19 is SARS Spike Glycoprotein Fragment synthetic DNA sequence with N-lined glycosylation site muations (2421 bp).

SEQ ID NO: 20 is SARS Spike glycoprotein Fragment I protein sequence without the N-linked glycosylation sites (807 amino acids encoded by SEQ ID NO: 19).

SEQ ID NO: 21 is SARS Spike glycoprotein Fragment II synthetic DNA sequence with N-linked glycosylation site mutations (744 bps).

SEQ ID NO: 22 is SARS Spike glycoprotein Fragment II protein sequence without the N-linked glycosylation sites (248 amino acids, encoded by SEQ ID 21)

SEQ ID NO: 23 is SARS Spike glycoprotein Fragment III synthetic DNA sequence with N-linked glycosylation site mutations (834 bps).

SEQ ID NO 24 is SARS Spike glycoprotein Fragment III protein sequence without the N-linked glycosylation sites (278 amino acids, encoded by SEQ ID NO: 23).

SEQ ID NO: 25 is SARS Spike glycoprotein Fragment IV synthetic DNA sequence with N-linked glycosylation site mutations (2034 bps).

SEQ ID NO: 26 is SARs Spike glycoprotein Fragment IV protein sequence without the N-linked glycosylation sites (678 amino acids, encoded by SEQ ID NO: 25).

### DETAILED DESCRIPTION OF THE INVENTION

This invention pertains to prevention and treatment of the disease caused by the SARS virus. This has been accomplished by the creation and synthesis of unique DNA sequences, unique polypeptides and proteins encoded by these sequences, vaccines and pharmaceutical compositions prepared using these unique DNA and polypeptide sequences, and monoclonal and polyclonal antibodies raised against these unique polypeptides. The strategy in generating these novel sequences was based upon the known sequence of a surface accessible protein referred to as the “spike protein of SARS-CoV, and to sub regions of that protein and polypeptides derived from that protein.

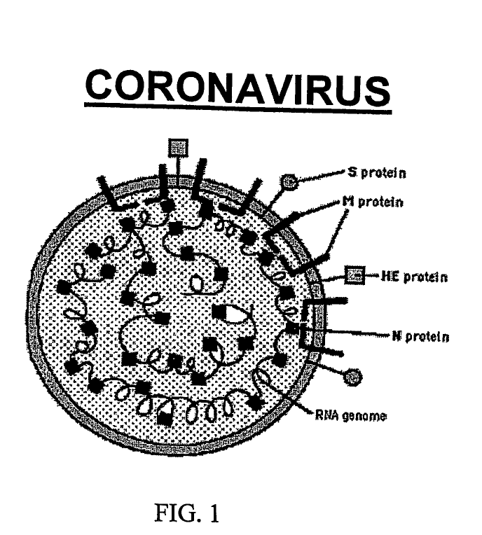

Furthermore, the applicants have used antibodies derived from these unique sequences to prepare therapeutics and diagnostics by providing and administering a polyclonal or monoclonal antibody against the S protein. The correlate of protective immunity for virtually all effective vaccines against viruses is the titer of antibodies that neutralizes virus invasion into cells and/or development of the virus within the cells. For antibodies to neutralize virus they in general have to be directed against proteins or glycoproteins that are accessible on the surface of viruses when they are extracellular. The “spike” (also called S or E2) glycoprotein of SARS-CoV is such a target for vaccine development (FIG. 1). This protein is even more attractive as a target because, it is thought to play a critical role in the pathogenesis of the infection and the disease. It is thought to mediate invasion of the virus into host (human) cells.

Alternative to production and use of the antibodies disclosed herein, the proteins and polypeptides of the present invention are use to stimulate the individual mammal or human to endogenously make antibodies after administration of a vaccine.

### The Spike Protein of SARS

The spike (S or E2) protein is encoded by 3768 base pairs (SEQ ID NO: 1), which encode a protein of 1256 amino acids (SEQ ID NO: 2). The gene sequence encoding the S protein, also called the Spike Protein and the E2 glycoprotein of SARS Coronavirus Urbani was extracted from the National Center for Biotechnology Information (NCBI) sequence database, accession number AY278741 (SEQ ID NO: 1). This sequence was submitted by Bellini, W. J. et al from the Division of Viral and Rickettsial Diseases, Centers for Disease Control and Prevention, 1600 Clifton RD, NE, Atlanta, Ga. 30333, USA.

### Analysis

The applicants analyzed the corresponding protein sequence (NCBI sequence accession number AAP13441)( SEQ ID NO: 2). Based on applicant's experience with identifying biologically active regions of proteins thought to mediate invasion into cells and to be good targets for antibody mediated protective immunity (Sim et al. 1994 Science 264:1941-44; Sim et al. 2001 Molecular Medicine 7:247-254), applicants selected a region of the SARS CoV spike (S or E2) protein to target. This region, which applicants have designated Region I spike protein SARS CoV, is encoded by 2421 base-paired nucleotides (bp 822 to 3243 of SEQ ID NO: 1) and codes for a protein from amino acid 275-1081 (807 amino acids) of the native protein (SEQ ID NO: 3).

Interestingly, Region I (SEQ ID NO: 3), derived from accession number AAP13441 from CDC was compared with the S-protein sequence from different AAP30030 from Beijing, AAP30713 from Hong Kong, AAP37017 from Taiwan, NP_{—}828851 from Canada. This 275-1081aa region of the S-protein is highly conserved. Among all the sequences published, there is a one amino acid conservative change at amino acid position 577 at which NP_{—}828851 is Alanine and the rest of the sequences are Serine.

### Potential N-Linked Glycosylation Sites of Region I

The S-protein sequence contains more than 22 potential N-linked glycosylation sites. According to experiments carried out in support of the present invention, N-glycosylation of such proteins reduces expression of the proteins and reduces immunogenicity for antibodies and thus, protective efficacy. Region I spike protein SARS CoV, the region from amino acid 275 to 1081 (SEQ ID NO: 3), was selected in part because of the paucity of N- glycosylation sites. The region was found to contain two interior domains with no potential N-glycosylation sites. Within the sequences flanking and intervening the two central domains, but included within amino acids 275-1081, there are 9 potential N-linked glycosylation sites (SEQ ID NO: 7).

All eukaryotic cells express N-linked glycoproteins. An N-linked gycosylation site requires a consensus sequence Asn-X-Ser/Thr in the primary sequence of the peptide at the putative site. Substitution of any of the amino acid residues in the glycosylation triplet will eliminate that site as a potential N-linked glycosylation site. In the embodiments which follow, alanine has been substituted for residues in the native sequence as indicated to eliminate potential N-linked glycosylation sites, but it will be understood by those skilled in the art that any amino acid substitution of either of the first and third residues in the triplet will accomplish the same goal.

Based on the Applicant's experience, the polypeptides of native regions I, II, III, IV are considered enhanced for expression of polypeptides and enhanced for reactive immunogenicity for antibody induction and thus, efficacious immunogenic regions of the Spike protein. This results from the infrequency of potential N-linked gycosylation sites identified by the Applicants. The polypeptides of novel regions I, II, III, IV, with potential N-linked glycosylation sites removed, are considered yet more expressible and immunogenic. Therefore, Regions I, II, III, IV polypeptides, with and without the gycosylation sites altered, are used to generate both polyclonal and monoclonal antibodies according to methods known in the art (Shinnick, T M, et al, Peptide-elicited protein-reactive antibodies in molecular biology and medicine (1984) J. Invest. Dermatol. (83) 112s-115s; Sim, K L, et al., Induction of Biologically Active Antibodies in Mice, rabbits, and Monkeys by Plasmodium falciparum EBA-175 Region II DNA vaccine (2001) Molecular Medicine (7) 247-254). These antibodies will be used for both diagnosis and treatment of SARS.

Inventively, the applicants defined a DNA sequence based on the known sequence of the Region I spike protein SARS CoV and then altered the known sequence to eliminate some or all (from 1 to 9) of these potential N-linked glycosylation sites. The methods for substitution of amino acids is well known in the art. (Merrifield, R. B. (1963). J. Am. Chem. Soc. 85, 2149-2154. Likewise, methods for producing mutant polynucleotide sequences are known in the art (see, for example, Ausubel et al [1997] Current Protocols in Molecular Biology, John Wiley & Sons, New York). In an embodiment, SEQ ID NO: 3 was altered at the amino acid residues corresponding to the following amino acids (as numbered in SEQ ID NO: 2): T³²⁰to A, N³³⁰to A, T³⁵⁹to A, S⁵⁹¹ to A, N⁶⁰²to A, N⁶⁹¹ to A, S⁷⁰¹to A, S⁷⁸⁵to A, T¹⁰⁵⁸ to A to remove the 9 potential N-linked glycosylation sites. This provided a unique polypeptide corresponding to Region I of the spike protein of SARS CoV native polypeptide in which the 9 identified potential N-linked glycosylation sites have been removed by the aforementioned Alanine substitutions. In this context the term “correspond” means that a DNA or polypeptide sequence is the same as a second sequence except for the specified substitutions. This novel polypeptide has been designated “RISPSCoVNNG” (SEQ ID NO: 4). The DNA sequence encoding SEQ ID NO: 4 with native SARS Coronavirus Urbani codon usage was designed and synthesized (R1SPSCoVNNG DNA—SEQ ID NO: 5). Methods of DNA synthesis are also well known in the art (Uhlmann E. (1988) Gene. November 15;71(1):29-40.

Applicant's extensive experience with expression of proteins in Pichia pastoris indicates that altering the codon usage (the degenerative flexibility of the DNA sequence code means multiple triplets code for the same amino acid) based on applicants' proprietary information leads to enhanced expression of the protein (Narum, D L, et al. Codon Optimization of Gene Fragments Encoding Plasmodium falciparum Merzoite Proteins Enhances DNA Vaccine Protein Expression and Immunogenicity in Mice. (2001) Infection and Immunity 69:7250-7253). Furthermore, this alteration of codon usage also enhances in vivo expression of proteins in DNA vaccines. We therefore used the Region I Spike Protein SARS CoV native no glycosylation (SEQ ID NO: 5) as the basis for creating a second synthetic gene in which we altered the sequence to optimize codon usage. This sequence is designated Region I Spike Protein SARS CoV co don optimized no glycosylation “RISPSCoVCONG” (SEQ ID NO: 6). It will be recognized by those skilled in the art that RISPSCoVCNNG and RISPSCoVCONG DNAs both code for the same polypeptide sequence which is designated the RISPSCoVCNNG (Region I) polypeptide (SEQ ID NO: 4).

These novel synthetic DNA sequences RISPSCoVNNG (SEQ ID NO: 5) and RISPSCoVCONG (SEQ ID NO: 6), and portions and sub regions thereof, are then used to construct novel DNA plasmid vaccines, recombinant virus vaccines, and recombinant bacteria vaccines, and to produce novel recombinant proteins and synthetic peptides that will be used as vaccines. Methods for expressing the polypeptide in a DNA plasmid, recombinant virus, recombinant bacteria, replicon, or other DNA or RNA based vaccine delivery system, or to produce a recombinant protein or synthetic peptide are well known in the art.

Methods of administration of peptide, DNA or RNA vaccines formed in accordance with the present invention are known in the art. As used herein, the term “administration” or “administering” refers to the process of delivering an agent to a patient, wherein the agent directly or indirectly increases the titer of anti-SARS antibody within the patient, along the lines described in the experimental rabbit model system described in Example 4 herein. The process of administration can be varied, depending on the agent, or agents, and the desired effect. Thus, the process of administration involves administering a selected immunogen of the invention to a patient in need of such treatment. Administration can be accomplished by any means appropriate for the therapeutic agent, for example, by parenteral, mucosal, pulmonary, topical, catheter-based, or oral means of delivery. Parenteral delivery can include for example, subcutaneous intravenous, intrauscular, intra-arterial, and injection into the tissue of an organ. Mucosal delivery can include, for example, intranasal delivery, preferably administered into the airways of a patient, i.e., nose, sinus, throat, lung, for example, as nose drops, by nebulization, vaporization, or other methods known in the art. Oral or intranasal delivery can include the administration of a propellant. Pulmonary delivery can include inhalation of the agent. Catheter-based delivery can include delivery by iontropheretic catheter-based delivery. Oral delivery can include delivery of a coated pill, or administration of a liquid by mouth. Administration can generally also include delivery with a pharmaceutically acceptable carrier, such as, for example, a buffer, a polypeptide, a peptide, a polysaccharide conjugate, a liposome, and/or a lipid, according to methods known in the art.

Compositions of the subject invention can be formulated according to known methods for preparing pharmaceutically useful compositions. Formulations are described in a number of sources which are well known and readily available to those skilled in the art. For example, Remington's Pharmaceutical Science (Martin E W [1995] Easton Pa., Mack Publishing Company, 19th Ed.) describes formulations which can be used in connection with the subject invention. Formulations suitable for parenteral administration include, for example, aqueous sterile injection solutions, which may contain antioxidants, buffers, bacteriostats, and solutes which render the formulation isotonic with the blood of the intended recipient; and aqueous and nonaqueous sterile suspensions which may include suspending agents and thickening agents. The formulations may be presented in unit-dose or multi-dose containers, for example sealed ampoules and vials, and may be stored in a freeze dried (lyophilized) condition requiring only the condition of the sterile liquid carrier, for example, water for injections, prior to use. Extemporaneous injection solutions and suspensions may be prepared from sterile powder, granules, tablets, etc. It should be understood that in addition to the ingredients particularly mentioned above, the formulations of the subject invention can include other agents conventional in the art having regard to the type of formulation in question.

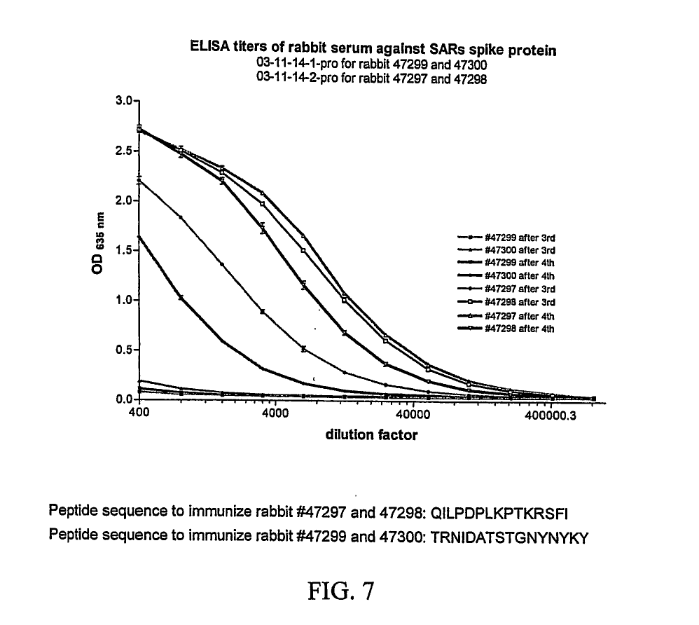

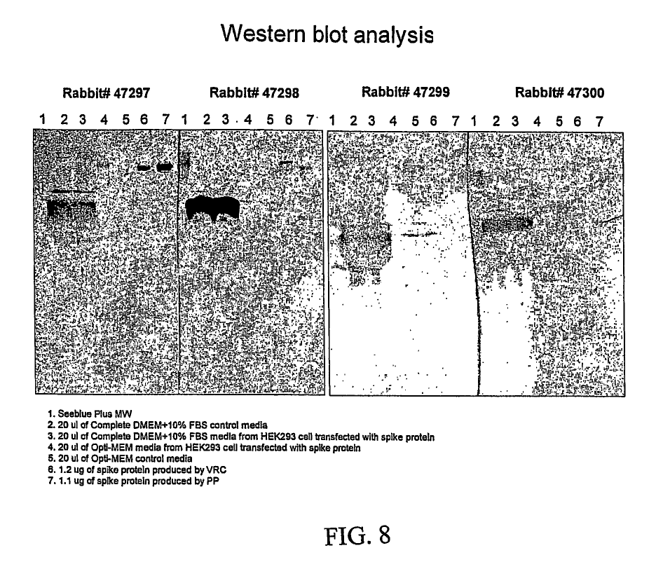

Therapeutically effective and optimal dosage ranges for vaccines and immunogens of the invention can be determined using methods known in the art. Guidance as to appropriate dosages to achieve an anti-viral effect is provided from the exemplified assays disclosed herein. More specifically, results from the immunization pattern described with reference to the rabbit animal model described in Example 4 and FIG. 7 herein can be extrapolated by persons having skill in the requisite art to provide a test vaccination schedule. Volunteer subjects or test animals can be inoculated with varying dosages at scheduled intervals and test blood samples can be evaluated for levels of antibody and/or SARS neutralizing activity present in the blood, using the guidance set forth in herein for evaluation of rabbit blood (for example, by Western blot analysis, as depicted in FIG. 8). Such results can be used to refine an optimized immunization dosage and schedule for effective immunization of mammalian, specifically human, subjects.

The polypeptides of native regions I, II, III, IV are considered enhanced immunogenic regions of the Spike protein due to the identified infrequency of potential N-linked glycosylation sites The polypeptides of novel regions I, II, III, IV, with potential N-linked glycosylation sites removed, are considered yet more immunogenic. Therefore, Regions I, II, III, IV polypeptides, with and without the glycosylation sites altered, are used to generated both polyclonal and monoclonal antibodies according to methods known in the art (Shinnick, T M, et al Peptide-elicited protein-reactive antibodies in molecular biology and medicine (1984) J. Invest. Dermatol. (83) 112s-115s; Sim, K L, et al., Induction of Biologically Active Antibodies in Mice, rabbits, and Monkeys by Plasmodium falciparum EBA-175 Region II DNA vaccine (2001) Molecular Medicine (7) 247-254). These antibodies can be used for both diagnosis and treatment of SARS. By way of example, it is contemplated that such antibodies may be used in kits and diagnostic tests for detecting the presence of SARS-CoV in bodily fluids, such as blood, blood fractions, saliva and the like. The anti-SARS antibodies may also be utilized in kits and tests used for environmental surveillance.

Both the foregoing description and the following Examples are exemplary and explanatory only and are not restrictive of the invention, as claimed. Moreover, the invention is not limited to the particular embodiments described, as such may, of course, vary. Further, the terminology used to describe particular embodiments is not intended to be limiting, since the scope of the present invention will be limited only by its claims.

With respect to ranges of values, the invention encompasses each intervening value between the upper and lower limits of the range to at least a tenth of the lower limit's unit, unless the context clearly indicates otherwise. Further, the invention encompasses any other stated intervening values. Moreover, the invention also encompasses ranges excluding either or both of the upper and lower limits of the range, unless specifically excluded from the stated range.

Unless defined otherwise, the meanings of all technical and scientific terms used herein are those commonly understood by one of ordinary skill in the art to which this invention belongs. One of ordinary skill in the art will also appreciate that any methods and materials similar or equivalent to those described herein can also be used to practice or test the invention. Further, all publications mentioned herein are incorporated by reference.

It must be noted that, as used herein and in the appended claims, the singular forms “a,” “or,” and “the” include plural referents unless the context clearly dictates otherwise. Further, all numbers expressing quantities of ingredients, reaction conditions, % purity, and so forth, used in the specification and claims, is modified by the term “about,” unless otherwise indicated. Accordingly, the numerical parameters set forth in the specification and claims are approximations that may vary depending upon the desired properties of the present invention. At the very least, and not as an attempt to limit the application of the doctrine of equivalents to the scope of, the claims, each numerical parameter should at least be construed in light of the number of reported significant digits, applying ordinary rounding techniques. Nonetheless, the numerical values set forth in the specific examples are reported as precisely as possible. Any numerical value, however, inherently contains certain errors from the standard deviation of its experimental measurement.

Obviously, many modifications and variations of the present invention are possible in light of the above teachings. It is therefore to be understood that, within the scope of the appended claims, the invention many be practiced otherwise than as specifically described.

The following examples further illustrate the invention. They are merely illustrative of the invention and disclose various beneficial properties of certain embodiments of the invention. These examples should not be construed as limiting the invention.

### EXAMPLES

### Example 1

### Spike Protein (SP) Fragment Cloning and Expression of Spike Protein in Pichia pastoris

### SP Region-I Gene:

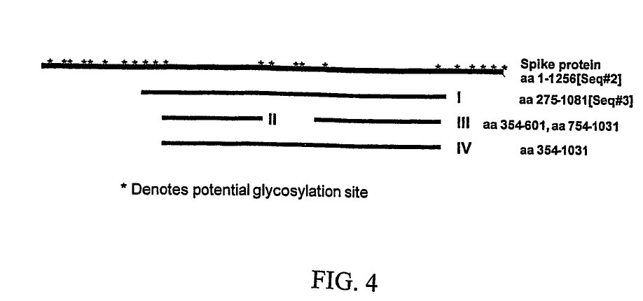

The 2421 bp gene fragment encoding amino acids 275-1081 (SEQ ID NO: 14) of the Spike protein (NCBI# AAP13441—SEQ ID NO: 3) with 9 potential N-linked glycosylation site mutations in which alanine was substituted as described above and XhoI and XbaI linker sequences at each end was synthesized. See FIG. 4.

### SP Region II Gene:

The 744 bp gene fragment encoding amino acids 354-601 (SEQ ID NO: 11) of the NCBI#AAP13441 Spike protein (SEQ ID NO: 3) was amplified by PCR using high fidelity DNA polymerase Vent with primers containing linkers with XhoI and XbaI restriction sites [oligo #1 (SEQ ID NO: 15) and #2 (SEQ ID NO: 16) using SP Region I plasmid as the DNA template (See Uhlmann E., above). This is shown in FIG. 4

### SP Region III Gene:

The 837 bp gene fragment encoding amino acids 754-1031 (SEQ ID NO: 12) of the NCBI#AAP13441 Spike protein (SEQ ID NO: 3) was amplified by PCR using high fidelity DNA polymerase Vent with primers containing linkers with XhoI and XbaI restriction sites [ oligo #3 (SEQ ID NO: 17) and oligo #4 (5′—SEQ ID NO: 18)] using SP Region I plasmid as the DNA template. (See Uhlmann E., above). This is shown in FIG. 4.

### SP Region IV Gene:

The 2034 bp gene fragment encoding aa 354-1031 (SEQ ID NO: 13) of the Spike protein (AAP13441; SEQ ID NO: 3) was amplified by PCR using high fidelity DNA polymerase Vent with primers containing linkers with XhoI and XbaI restriction sites [ oligo #1 (SEQ ID NO: 15) and oligo #4 ( SEQ ID NO: 18)] using SP Region I plasmid as the DNA template. (See Uhlmann E., above). This is shown in FIG. 4.

All 4 gene fragments were gel purified, digested with XhoI and XbaI restriction enzymes, ligated into the XhoI/XbaI sites of pPICZaA separately. Applicant's cloning strategy utilized the fact that the target gene is driven by the powerful alcohol oxidase AOX1 promoter that is inducible with methanol. Applicant's cloning strategy also utilizes the alpha factor secretion signal of native yeast origin, ultimately used for secreting each recombinant Spike protein fragment into the culture media.

Each ligation mix was transformed into Top10 E. coli strain and the plasmid DNAs were analyzed by restriction mapping. Two of the plasmids with the correct gene sequences for each construct were linearized and transformed into P. pastoris host strain (X33). Three transformants were selected for Zeocin resistance over increasing Zeocin concentrations for each plasmid. Six transformants were picked and screened for their protein expression levels for each construct. Single colonies derived from those transformants were grown in BMGY/BMMY medium (100 mM Potassium phosphate, pH 5.6, 1% yeast extract, 2% peptone, 1.34% yeast nitrogen base without amino acids, 4×10⁻⁵% biotin, 1% glycerol for BMGY and 0.5% methanol for BMMY) and induced at both 24° C. and 30° C. to test their productivities.

The supernatants from 24 h, 48 h, 72 h, 96 h and 120 h post-induction time points were analyzed on both Coomassie stained SDS-PAGE gels and immunoblots (using sera generated from immunization of synthetic peptides in rabbits).

### Example 2

### Preparation of the P. pastoris Clone Glycerol Cell Bank Stock

A single colony from the plate with Zeocin plate will be inoculated into BMGY and grown overnight at 30° C. at 250 rpm. Sterility of the culture will be examined by light microscopy (400× magnification). A glycerol stock (P1) will be prepared in 15% glycerol and stored at −70° C. A P2 glycerol stock will be prepared by inoculating 100 μl of P1 stock into 100 ml of BMGY and grown overnight at 30° C. at 250 rpm and saved in a final of 15% glycerol and stored at −70° C.

### Example 3

### Recombinant Protein Expression by Shake-Flask Fermentation

One vial of P. pastoris clone P2 glycerol stock is inoculated into 100 ml of BMGY in a 500 ml flask and incubated at 250 rpm at 30° C. for ˜24 h to OD₆₀₀ 5-10. 15 ml of this culture is inoculated into 1 L BMGY in a 4 L-baffled flask and grown at the same condition for another 24 h. The culture is centrifuged at 5000 rpm in a GSA rotor for 5 min and the cell pellets is resuspended in BMMY at 80 g/L cell density. The concentrated cell culture is placed into a 4 L-baffled flask and induced at the selected temperature with 250 rpm agitation. The culture is fed with 2% methanol every 24 h and the supernatant is collected at the selected post-induction time point by centrifugation at 5000 rpm for 5 minutes, and stored at −70° C.

### Example 4

### T Cell Epitope Prediction

Using a T cell epitope prediction algorithm applicants have predicted 9 HLA A0201, A0301 or B0801 (class I) and 6 DRB1 (class II) T-cell epitopes in this region of the SARS CoV spike protein. The sequences of thirteen of the 15 epitopes were completely conserved in the sequences of 13 isolates of SARS CoV that we were able to compare. These data indicate that, 1) There are adequate T-cell helper epitopes in the protein that we are working on to insure a broad antibody response in immunized humans, and 2) There are adequate CD8 T-cell epitopes to insure that at least 90% of the world's population will be able to mount CD8 T-cell responses to this portion of the protein. Such data will provide the foundation for parallel T-cell studies as we move forward with vaccine development.

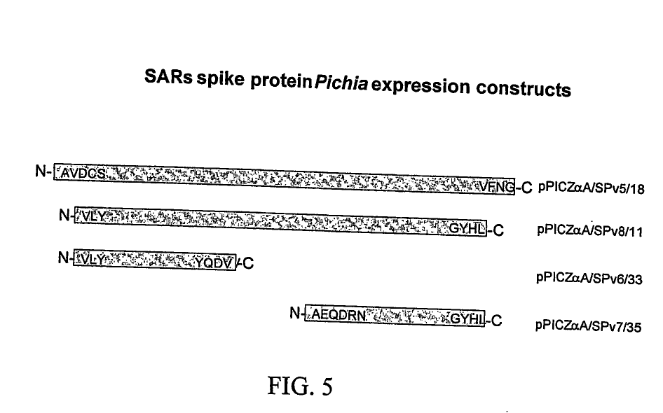

Applicants selected potential B-cell epitopes and synthesized peptides from native SEQ ID NO: 3. These peptides were conjugated to Keyhole limpet hemocyanin and used to immunize rabbits with Freund's adjuvant. The rabbits were bled 2 weeks after the third dose for sera containing polyclonal antibodies against the peptides. The titers found in the rabbits against the peptides are shown in FIG. 4. Immunoblots (Westerns) show that the polyclonal sera are specific against the Spike protein (FIG. 5).

In the foregoing, the present invention has been described with reference to suitable embodiments, but these embodiments are only for purposes of understanding the invention and various alterations or modifications are possible so long as the present invention does not deviate from the claims that follow.

All references cited, as well as the provisional application upon which this application is based, are incorporated herein by reference. All nucleotide and amino acid sequences mentioned herein or appended hereto are incorporated herein by reference.

What is claimed is:

1. An isolated DNA molecule comprising a nucleotide sequence of at least 600 consecutive base pairs of SEQ ID NO: 5.

2. An isolated DNA molecule having a nucleotide sequence encoding a Region I polypeptide comprising the sequence SEQ ID NO: 4.

3. The DNA molecule of claim 2, wherein said nucleotide sequence is operatively linked in a replicon.

4. The DNA molecule of claim 2, wherein said nucleotide sequence encodes a Region II polypeptide having the sequence SEQ ID NO: 8.

5. The DNA molecule of claim 4, wherein said nucleotide sequence is operatively linked in a replicon.

6. The DNA molecule of claim 2, wherein said nucleotide sequence encodes a Region III polypeptide having the sequence SEQ ID NO: 9.

7. The DNA molecule of claim 6 wherein said nucleotide sequence is operatively linked in a replicon.

8. The DNA molecule of claim 2, wherein said nucleotide sequence encodes encoding a Region IV polypeptide having the sequence SEQ ID NO: 10.

9. The DNA molecule of claim 8, wherein said nucleotide sequence is operatively linked in a replicon.

10. An isolated DNA molecule comprising a nucleotide sequence of at least 600 consecutive base pairs of SEQ ID NO: 6 or SEQ ID NO: 19.

11. The DNA molecule of claim 10, wherein said nucleotide sequence encodes a Region I polypeptide having the sequence SEQ ID NO: 5 or SEQ ID NO: 20.

12. The DNA molecule of claim 11, wherein said nucleotide sequence is operatively linked in a replicon.

13. The DNA molecule of claim 10, wherein said nucleotide sequence encodes a Region II polypeptide having the sequence SEQ ID NO: 8 or SEQ ID NO: 22.

14. The DNA molecule of claim 13, wherein said nucleotide sequence is operatively linked in a replicon.

15. The DNA molecule of claim 10, wherein said nucleotide sequence encodes a Region III polypeptide having the sequence SEQ ID NO: 9 or SEQ ID NO: 24.

16. The DNA molecule of claim 15 wherein said nucleotide sequence is operatively linked in a replicon.

17. The DNA molecule of claim 10, wherein said nucleotide sequence encodes a Region IV polypeptide having the sequence SEQ ID NO: 10 or SEQ ID NO: 26.

18. The DNA molecule of claim 17, wherein said nucleotide sequence is operatively linked in a replicon.

19. An isolated polypeptide molecule comprising at least 200 consecutive amino acid residues of SEQ ID NO: 3, wherein one or more potential N-linked glycosylation sites in said polypeptide are eliminated by amino acid substitution, said substitution sites selected from the group consisting of T³²⁰, N³³⁰, T³⁵⁹, S⁵⁹¹, N⁶⁰², N⁶⁹¹, S⁷⁰¹, S⁷⁸⁵, and T¹⁰⁵⁸.

20. The polypeptide of claim 19, comprising a Region I peptide having the sequence SEQ ID NO: 14 or SEQ ID NO: 20.

21. The polypeptide of claim 20, wherein the potential N-linked glycosylation sites in said polypeptide are eliminated by substitution with alanine.

22. The polypeptide of claim 19, comprising the amino acid sequence SEQ ID NO: 11, wherein alanine is substituted for T ³⁵⁹ and S⁵⁹¹.

23. The polypeptide of claim 19 comprising the amino acid sequence SEQ ID NO: 12, wherein alanine is substituted for S⁷⁸⁵

24. The polypeptide of claim 19, comprising the amino acid sequence SEQ ID NO: 13, wherein alanine is substituted for T³⁵⁹, S⁵⁹¹, N⁶⁰², N⁶⁹¹, S⁷⁰¹ and S⁷⁸⁵

25. A vaccine for the prevention, attenuation or treatment of Severe Acute Respiratory Syndrome (SARS) in a mammal comprising polynucleotide having a sequence of at least 600 consecutive base pairs of SEQ ID NO: 6 or SEQ ID NO: 19 and a carrier.

26. The vaccine of claim 25, wherein said polynucleotide encodes a polypeptide comprising the sequence SEQ ID NO: 4 or SEQ ID NO: 20.

27. The vaccine of claim 25, wherein said polynucleotide encodes a peptide comprising the sequence SEQ ID NO: 8 or SEQ ID NO: 22.

28. The vaccine of claim 25, wherein said polynucleotide encodes a peptide comprising the sequence SEQ ID NO: 9 or SEQ ID NO. 24.

29. The vaccine of claim 25, wherein said polynucleotide encodes a polypeptide comprising the sequence SEQ ID NO: 10 or SEQ ID NO: 26.

30. A vaccine for the prevention, attenuation or treatment of Severe Acute Respiratory Syndrome (SARS) in a mammal comprising a polypeptide corresponding to SEQ ID NO: 3, said polypeptide comprising at least 200 residues wherein one or more potential N-linked glycosylation sites are optionally substituted to block glycosylation, and a carrier.

31. The vaccine of claim 30, wherein said glycosylation sites are selected from the group consisting of T³²⁰, N³³⁰, T³⁵⁹, S⁵⁹¹, N⁶⁰², N⁶⁹¹, S⁷⁰¹, S⁷⁸⁵, and T¹⁰⁵⁸.

32. The vaccine of claim 31, wherein at least one potential N-linked glycosylation sites in said polypeptide is substituted with alanine.

33. The vaccine of claim 32, wherein alanine is substituted for T³⁵⁹ and S⁵⁹¹.

34. The vaccine of claim 32, wherein alanine is substituted for S⁷⁸⁵.

35. The vaccine of claim 32, wherein alanine is substituted for T³⁵⁹, S⁵⁹¹, N⁶⁰², N⁶⁹¹, S⁷⁰¹ and S⁷⁸⁵.

36. An antibody against a polypeptide molecule of claim 19.

37. The antibody of claim 36 wherein said antibody is polyclonal.

38. The antibody of claim 36 wherein said antibody is monoclonal.

39. The antibody of claim 37 or claim 38 wherein said polypeptide comprises the a Region I peptide having SEQ ID NO: 14 or SEQ ID NO: 20.

40. The antibody of claim 39 wherein one or more potential N-linked glycosylation sites in said polypeptide are eliminated by amino acid substitution.

41. The antibody of claim 37 or 38 wherein alanine is substituted for T³⁵⁹ and S⁵⁹¹ in said polypeptide.

42. The antibody of claim 37 or 38 wherein alanine is substituted for S⁷⁸⁵ in said polypeptide.

43. The antibody of claim 37 or 38 wherein alanine is substituted for T³⁵⁹, S⁵⁹¹, N⁶⁰², N⁶⁹¹, S⁷⁰¹ and S⁷⁸⁵ in said polypeptide.

44. An isolated DNA molecule encoding a polypeptide comprising at least 200 consecutive amino acid residues of SEQ ID NO: 3.

45. The DNA molecule of claim 44 encoding all of Seq ID NO:3.

46. A isolated polypeptide molecule comprising at least 200 consecutive amino acid residues of a polypeptide having the sequence SEQ ID NO: 3.

47. The polypeptide of claim 46 comprising the entire SEQ ID NO: 3.

48. A vaccine for the prevention, attenuation or treatment of Severe Acute Respiratory Syndrome (SARS) in a mammal comprising a nucleotide sequence encoding at least 200 amino acid residues of SEQ ID NO: 3.

49. A vaccine for the prevention, attenuation or treatment of Severe Acute Respiratory Syndrome (SARS) in a mammal comprising a polypeptide containing at least 200 amino acid residues of SEQ ID NO: 3.

50. A DNA molecule encoding a Region I polypeptide fragment.

51. A DNA molecule encoding a Region II polypeptide fragment.

52. A DNA molecule encoding a Region III polypeptide fragment.

53. A DNA molecule encoding a Region IV polypeptide fragment.

54. The DNA molecule of claims 50-53, wherein the fragment is a native fragment.

55. The DNA molecule of any of claims 50-53, wherein the fragment comprises at least one eliminated glycosylation site.

56. The DNA of any of claims 50-55, wherein said DNA is codon-optimized.

57. A polypeptide expressed by a DNA molecule of any of claims 50-55.

58. A vaccine comprising a DNA molecule of any of claims 50-55.

59. A vaccine comprising a polypeptide of claim 57.

60. An antibody to a polypeptide of claim 57.


SEQUENCE LISTING (33 sequences)

SEQ ID NO: 1 - DNA, 3768 bp, SARS Coronavirus urbani
atgtttattttcttattatttcttactctcactagtggtagtgaccttgaccggtgcaccacttttgatgatgttcaagctcctaattacactcaacatacttcatctatgaggggggtttactatcctgatgaaatttttagatcagacactctttatttaactcaggatttatttcttccattttattctaatgttacagggtttcatactattaatcatacgtttggcaaccctgtcataccttttaaggatggtatttattttgctgccacagagaaatcaaatgttgtccgtggttgggtttttggttctaccatgaacaacaagtcacagtcggtgattattattaacaattctactaatgttgttatacgagcatgtaactttgaattgtgtgacaaccctttctttgctgtttctaaacccatgggtacacagacacatactatgatattcgataatgcatttaattgcactttcgagtacatatctgatgccttttcgcttgatgtttcagaaaagtcaggtaattttaaacacttacgagagtttgtgtttaaaaataaagatgggtttctctatgtttataagggctatcaacctatagatgtagttcgtgatctaccttctggttttaacactttgaaacctatttttaagttgcctcttggtattaacattacaaattttagagccattcttacagccttttcacctgctcaagacatttggggcacgtcagctgcagcctattttgttggctatttaaagccaactacatttatgctcaagtatgatgaaaatggtacaatcacagatgctgttgattgttctcaaaatccacttgctgaactcaaatgctctgttaagagctttgagattgacaaaggaatttaccagacctctaatttcagggttgttccctcaggagatgttgtgagattccctaatattacaaacttgtgtccttttggagaggtttttaatgctactaaattcccttctgtctatgcatgggagagaaaaaaaatttctaattgtgttgctgattactctgtgctctacaactcaacatttttttcaacctttaagtgctatggcgtttctgccactaagttgaatgatctttgcttctccaatgtctatgcagattcttttgtagtcaagggagatgatgtaagacaaatagcgccaggacaaactggtgttattgctgattataattataaattgccagatgatttcatgggttgtgtccttgcttggaatactaggaacattgatgctacttcaactggtaattataattataaatataggtatcttagacatggcaagcttaggccctttgagagagacatatctaatgtgcctttctcccctgatggcaaaccttgcaccccacctgctcttaattgttattggccattaaatgattatggtttttacaccactactggcattggctaccaaccttacagagttgtagtactttcttttgaacttttaaatgcaccggccacggtttgtggaccaaaattatccactgaccttattaagaaccagtgtgtcaattttaattttaatggactcactggtactggtgtgttaactccttcttcaaagagatttcaaccatttcaacaatttggccgtgatgtttctgatttcactgattccgttcgagatcctaaaacatctgaaatattagacatttcaccttgctcttttgggggtgtaagtgtaattacacctggaacaaatgcttcatctgaagttgctgttctatatcaagatgttaactgcactgatgtttctacagcaattcatgcagatcaactcacaccagcttggcgcatatattctactggaaacaatgtattccagactcaagcaggctgtcttataggagctgagcatgtcgacacttcttatgagtgcgacattcctattggagctggcatttgtgctagttaccatacagtttctttattacgtagtactagccaaaaatctattgtggcttatactatgtctttaggtgctgatagttcaattgcttactctaataacaccattgctatacctactaacttttcaattagcattactacagaagtaatgcctgtttctatggctaaaacctccgtagattgtaatatgtacatctgcggagattctactgaatgtgctaatttgcttctccaatatggtagcttttgcacacaactaaatcgtgcactctcaggtattgctgctgaacaggatcgcaacacacgtgaagtgttcgctcaagtcaaacaaatgtacaaaaccccaactttgaaatattttggtggttttaatttttcacaaatattacctgaccctctaaagccaactaagaggtcttttattgaggacttgctctttaataaggtgacactcgctgatgctggcttcatgaagcaatatggcgaatgcctaggtgatattaatgctagagatctcatttgtgcgcagaagttcaatggacttacagtgttgccacctctgctcactgatgatatgattgctgcctacactgctgctctagttagtggtactgccactgctggatggacatttggtgctggcgctgctcttcaaataccttttgctatgcaaatggcatataggttcaatggcattggagttacccaaaatgttctctatgagaaccaaaaacaaatcgccaaccaatttaacaaggcgattagtcaaattcaagaatcacttacaacaacatcaactgcattgggcaagctgcaagacgttgttaaccagaatgctcaagcattaaacacacttgttaaacaacttagctctaattttggtgcaatttcaagtgtgctaaatgatatcctttcgcgacttgataaagtcgaggcggaggtacaaattgacaggttaattacaggcagacttcaaagccttcaaacctatgtaacacaacaactaatcagggctgctgaaatcagggcttctgctaatcttgctgctactaaaatgtctgagtgtgttcttggacaatcaaaaagagttgacttttgtggaaagggctaccaccttatgtccttcccacaagcagccccgcatggtgttgtcttcctacatgtcacgtatgtgccatcccaggagaggaacttcaccacagcgccagcaatttgtcatgaaggcaaagcatacttccctcgtgaaggtgtttttgtgtttaatggcacttcttggtttattacacagaggaacttcttttctccacaaataattactacagacaatacatttgtctcaggaaattgtgatgtcgttattggcatcattaacaacacagtttatgatcctctgcaacctgagctcgactcattcaaagaagagctggacaagtacttcaaaaatcatacatcaccagatgttgatcttggcgacatttcaggcattaacgcttctgtcgtcaacattcaaaaagaaattgaccgcctcaatgaggtcgctaaaaatttaaatgaatcactcattgaccttcaagaattgggaaaatatgagcaatatattaaatggccttggtatgtttggctcggcttcattgctggactaattgccatcgtcatggttacaatcttgctttgttgcatgactagttgttgcagttgcctcaagggtgcatgctcttgtggttcttgctgcaagtttgatgaggatgactctgagccagttctcaagggtgtcaaattacattacacataa

SEQ ID NO: 2 - protein, 1255 aa, SARS Coronavirus urbani
MFIFLLFLTLTSGSDLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCSFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYIKWPWYVWLGFIAGLIAIVMVTILLCCMTSCCSCLKGACSCGSCCKFDEDDSEPVLKGVKLHYT

SEQ ID NO: 3 - protein, 807 aa, SARS Coronavirus urbani
AVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCSFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNG

SEQ ID NO: 4 - protein, 807 aa, SARS Coronavirus urbani
AVDCSQNPLAELKCSVKSFEIDKGIYQTSNFRVVPSGDVVRFPNIANLCPFGEVFAATKFPSVYAWERKKISNCVADYSVLYNSAFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCSFGGVSVITPGTNAASEVAVLYQDVACTDVSTAIHADQLTPAWRIYSTGNNVFQTQAGCLIGAEHVDTSYECDIPIGAGICASYHTVSLLRSTSQKSIVAYTMSLGADSSIAYSANTIAIPTNFAISITTEVMPVSMAKTSVDCNMYICGDSTECANLLLQYGSFCTQLNRALSGIAAEQDRNTREVFAQVKQMYKTPTLKYFGGFNFAQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFATAPAICHEGKAYFPREGVFVFNG

SEQ ID NO: 5 - DNA, 2421 bp, SARS Coronavirus urbani
gctgttgattgttctcaaaatccacttgctgaactcaaatgctctgttaagagctttgagattgacaaaggaatttaccagacctctaatttcagggttgttccctcaggagatgttgtgagattccctaatattgcaaacttgtgtccttttggagaggtttttgccgctactaaattcccttctgtctatgcatgggagagaaaaaaaatttctaattgtgttgctgattactctgtgctctacaactcagcatttttttcaacctttaagtgctatggcgtttctgccactaagttgaatgatctttgcttctccaatgtctatgcagattcttttgtagtcaagggagatgatgtaagacaaatagcgccaggacaaactggtgttattgctgattataattataaattgccagatgatttcatgggttgtgtccttgcttggaatactaggaacattgatgctacttcaactggtaattataattataaatataggtatcttagacatggcaagcttaggccctttgagagagacatatctaatgtgcctttctcccctgatggcaaaccttgcaccccacctgctcttaattgttattggccattaaatgattatggtttttacaccactactggcattggctaccaaccttacagagttgtagtactttcttttgaacttttaaatgcaccggccacggtttgtggaccaaaattatccactgaccttattaagaaccagtgtgtcaattttaattttaatggactcactggtactggtgtgttaactccttcttcaaagagatttcaaccatttcaacaatttggccgtgatgtttctgatttcactgattccgttcgagatcctaaaacatctgaaatattagacatttcaccttgctcttttgggggtgtaagtgtaattacacctggaacaaatgctgcatctgaagttgctgttctatatcaagatgttgcctgcactgatgtttctacagcaattcatgcagatcaactcacaccagcttggcgcatatattctactggaaacaatgtattccagactcaagcaggctgtcttataggagctgagcatgtcgacacttcttatgagtgcgacattcctattggagctggcatttgtgctagttaccatacagtttctttattacgtagtactagccaaaaatctattgtggcttatactatgtctttaggtgctgatagttcaattgcttactctgccaacaccattgctatacctactaactttgcaattagcattactacagaagtaatgcctgtttctatggctaaaacctccgtagattgtaatatgtacatctgcggagattctactgaatgtgctaatttgcttctccaatatggtagcttttgcacacaactaaatcgtgcactctcaggtattgctgctgaacaggatcgcaacacacgtgaagtgttcgctcaagtcaaacaaatgtacaaaaccccaactttgaaatattttggtggttttaattttgcacaaatattacctgaccctctaaagccaactaagaggtcttttattgaggacttgctctttaataaggtgacactcgctgatgctggcttcatgaagcaatatggcgaatgcctaggtgatattaatgctagagatctcatttgtgcgcagaagttcaatggacttacagtgttgccacctctgctcactgatgatatgattgctgcctacactgctgctctagttagtggtactgccactgctggatggacatttggtgctggcgctgctcttcaaataccttttgctatgcaaatggcatataggttcaatggcattggagttacccaaaatgttctctatgagaaccaaaaacaaatcgccaaccaatttaacaaggcgattagtcaaattcaagaatcacttacaacaacatcaactgcattgggcaagctgcaagacgttgttaaccagaatgctcaagcattaaacacacttgttaaacaacttagctctaattttggtgcaatttcaagtgtgctaaatgatatcctttcgcgacttgataaagtcgaggcggaggtacaaattgacaggttaattacaggcagacttcaaagccttcaaacctatgtaacacaacaactaatcagggctgctgaaatcagggcttctgctaatcttgctgctactaaaatgtctgagtgtgttcttggacaatcaaaaagagttgacttttgtggaaagggctaccaccttatgtccttcccacaagcagccccgcatggtgttgtcttcctacatgtcacgtatgtgccatcccaggagaggaacttcgccacagcgccagcaatttgtcatgaaggcaaagcatacttccctcgtgaaggtgtttttgtgtttaatggc

SEQ ID NO: 6 - DNA, 2421 bp, SARS Coronavirus urbani
gccgtggactgctcccagaaccccctggccgagctgaagtgctccgtgaagtccttcgagatcgacaagggcatctaccagacctccaacttccgcgtggtgccctccggcgacgtggtgcgcttccccaacatcgccaacctgtgccccttcggcgaggtgttcgccgccaccaagttcccctccgtgtacgcctgggagcgcaagaagatctccaactgcgtggccgactactccgtgctgtacaactccgccttcttctccaccttcaagtgctacggcgtgtccgccaccaagctgaacgacctgtgcttctccaacgtgtacgccgactccttcgtggtgaagggcgacgacgtgcgccagatcgcccccggccagaccggcgtgatcgccgactacaactacaagctgcccgacgacttcatgggctgcgtgctggcctggaacacccgcaacatcgacgccacctccaccggcaactacaactacaagtaccgctacctgcgccacggcaagctgcgccccttcgagcgcgacatctccaacgtgcccttctcccccgacggcaagccctgcaccccccccgccctgaactgctactggcccctgaacgactacggcttctacaccaccaccggcatcggctaccagccctaccgcgtggtggtgctgtccttcgagctgctgaacgcccccgccaccgtgtgcggccccaagctgtccaccgacctgatcaagaaccagtgcgtgaacttcaacttcaacggcctgaccggcaccggcgtgctgaccccctcctccaagcgcttccagcccttccagcagttcggccgcgacgtgtccgacttcaccgactccgtgcgcgaccccaagacctccgagatcctggacatctccccctgctccttcggcggcgtgtccgtgatcacccccggcaccaacgccgcctccgaggtggccgtgctgtaccaggacgtggcctgcaccgacgtgtccaccgccatccacgccgaccagctgacccccgcctggcgcatctactccaccggcaacaacgtgttccagacccaggccggctgcctgatcggcgccgagcacgtggacacctcctacgagtgcgacatccccatcggcgccggcatctgcgcctcctaccacaccgtgtccctgctgcgctccacctcccagaagtccatcgtggcctacaccatgtccctgggcgccgactcctccatcgcctactccgccaacaccatcgccatccccaccaacttcgccatctccatcaccaccgaggtgatgcccgtgtccatggccaagacctccgtggactgcaacatgtacatctgcggcgactccaccgagtgcgccaacctgctgctgcagtacggctccttctgcacccagctgaaccgcgccctgtccggcatcgccgccgagcaggaccgcaacacccgcgaggtgttcgcccaggtgaagcagatgtacaagacccccaccctgaagtacttcggcggcttcaacttcgcccagatcctgcccgaccccctgaagcccaccaagcgctccttcatcgaggacctgctgttcaacaaggtgaccctggccgacgccggcttcatgaagcagtacggcgagtgcctgggcgacatcaacgcccgcgacctgatctgcgcccagaagttcaacggcctgaccgtgctgccccccctgctgaccgacgacatgatcgccgcctacaccgccgccctggtgtccggcaccgccaccgccggctggaccttcggcgccggcgccgccctgcagatccccttcgccatgcagatggcctaccgcttcaacggcatcggcgtgacccagaacgtgctgtacgagaaccagaagcagatcgccaaccagttcaacaaggccatctcccagatccaggagtccctgaccaccacctccaccgccctgggcaagctgcaggacgtggtgaaccagaacgcccaggccctgaacaccctggtgaagcagctgtcctccaacttcggcgccatctcctccgtgctgaacgacatcctgtcccgcctggacaaggtggaggccgaggtgcagatcgaccgcctgatcaccggccgcctgcagtccctgcagacctacgtgacccagcagctgatccgcgccgccgagatccgcgcctccgccaacctggccgccaccaagatgtccgagtgcgtgctgggccagtccaagcgcgtggacttctgcggcaagggctaccacctgatgtccttcccccaggccgccccccacggcgtggtgttcctgcacgtgacctacgtgccctcccaggagcgcaacttcgccaccgcccccgccatctgccacgagggcaaggcctacttcccccgcgagggcgtgttcgtgttcaacggc

SEQ ID NO: 7 - protein, 807 aa, SARS Coronavirus urbani; identical to SEQ ID NO: 3

SEQ ID NO: 8 - protein, 248 aa, SARS Coronavirus urbani
VLYNSAFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCSFGGVSVITPGTNAASEVAVLYQDV

SEQ ID NO: 9 - protein, 278 aa, SARS Coronavirus urbani
AEQDRNTREVFAQVKQMYKTPTLKYFGGFNFAQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHL

SEQ ID NO: 10 - protein, 678 aa, SARS Coronavirus urbani
VLYNSAFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCSFGGVSVITPGTNAASEVAVLYQDVACTDVSTAIHADQLTPAWRIYSTGNNVFQTQAGCLIGAEHVDTSYECDIPIGAGICASYHTVSLLRSTSQKSIVAYTMSLGADSSIAYSANTIAIPTNFAISITTEVMPVSMAKTSVDCNMYICGDSTECANLLLQYGSFCTQLNRALSGIAAEQDRNTREVFAQVKQMYKTPTLKYFGGFNFAQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHL

SEQ ID NO: 11 - protein, 248 aa, SARS Coronavirus urbani
VLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCSFGGVSVITPGTNASSEVAVLYQDV

SEQ ID NO: 12 - protein, 278 aa, SARS Coronavirus urbani
AEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHL

SEQ ID NO: 13 - protein, 678 aa, SARS Coronavirus urbani
VLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCSFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHL

SEQ ID NO: 14 - protein, 757 aa, SARS Coronavirus urbani
AVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCSFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHL

SEQ ID NO: 15 - DNA, 36 bp, Artificial Sequence
  Description of Artificial Sequence Synthetic oligonucleotide
tcgctcgagaaaagagtgctctacaactcagcattt

SEQ ID NO: 16 - DNA, 33 bp, Artificial Sequence
  Description of Artificial Sequence Synthetic oligonucleotide
atctctagattaaacatcttgatatagaacagc

SEQ ID NO: 17 - DNA, 36 bp, Artificial Sequence
  Description of Artificial Sequence Synthetic oligonucleotide
tcgctcgagaaaagagctgaacaggatcgcaacaca

SEQ ID NO: 18 - DNA, 30 bp, Artificial Sequence
  Description of Artificial Sequence Synthetic oligonucleotide
atctctagattaaaggtggtagccctttcc

SEQ ID NO: 19 - DNA, 2421 bp, SARS Coronavirus urbani
gccgtggactgctcccagaacccactggccgagctgaagtgctccgtgaagtccttcgagatcgacaagggcatctaccagacctccaacttccgcgtggtgccatccggcgacgtggtgcgcttcccaaacatcgccaacctgtgcccattcggcgaggtgttcgccgccaccaagttcccatccgtgtacgcctgggagcgcaagaagatctccaactgcgtggccgactactccgtgctgtacaactccgccttcttctccaccttcaagtgctacggcgtgtccgccaccaagctgaacgacctgtgcttctccaacgtgtacgccgactccttcgtggtgaagggcgacgacgtgcgccagatcgccccaggccagaccggcgtgatcgccgactacaactacaagctgccagacgacttcatgggctgcgtgctggcctggaacacccgcaacatcgacgccacctccaccggcaactacaactacaagtaccgctacctgcgccacggcaagctgcgcccattcgagcgcgacatctccaacgtgccattctccccagacggcaagccatgcaccccaccagccctgaactgctactggccactgaacgactacggcttctacaccaccaccggcatcggctaccagccataccgcgtggtggtgctgtccttcgagctgctgaacgccccagccaccgtgtgcggcccaaagctgtccaccgacctgatcaagaaccagtgcgtgaacttcaacttcaacggcctgaccggcaccggcgtgctgaccccatcctccaagcgcttccagccattccagcagttcggccgcgacgtgtccgacttcaccgactccgtgcgcgacccaaagacctccgagatcctggacatctccccatgctccttcggcggcgtgtccgtgatcaccccaggcaccaacgccgcctccgaggtggccgtgctgtaccaggacgtggcctgcaccgacgtgtccaccgccatccacgccgaccagctgaccccagcctggcgcatctactccaccggcaacaacgtgttccagacccaggccggctgcctgatcggcgccgagcacgtggacacctcctacgagtgcgacatcccaatcggcgccggcatctgcgcctcctaccacaccgtgtccctgctgcgctccacctcccagaagtccatcgtggcctacaccatgtccctgggcgccgactcctccatcgcctactccgccaacaccatcgccatcccaaccaacttcgccatctccatcaccaccgaggtgatgccagtgtccatggccaagacctccgtggactgcaacatgtacatctgcggcgactccaccgagtgcgccaacctgctgctgcagtacggctccttctgcacccagctgaaccgcgccctgtccggcatcgccgccgagcaggaccgcaacacccgcgaggtgttcgcccaggtgaagcagatgtacaagaccccaaccctgaagtacttcggcggcttcaacttcgcccagatcctgccagacccactgaagccaaccaagcgctccttcatcgaggacctgctgttcaacaaggtgaccctggccgacgccggcttcatgaagcagtacggcgagtgcctgggcgacatcaacgcccgcgacctgatctgcgcccagaagttcaacggcctgaccgtgctgccaccactgctgaccgacgacatgatcgccgcctacaccgccgccctggtgtccggcaccgccaccgccggctggaccttcggcgccggcgccgccctgcagatcccattcgccatgcagatggcctaccgcttcaacggcatcggcgtgacccagaacgtgctgtacgagaaccagaagcagatcgccaaccagttcaacaaggccatctcccagatccaggagtccctgaccaccacctccaccgccctgggcaagctgcaggacgtggtgaaccagaacgcccaggccctgaacaccctggtgaagcagctgtcctccaacttcggcgccatctcctccgtgctgaacgacatcctgtcccgcctggacaaggtggaggccgaggtgcagatcgaccgcctgatcaccggccgcctgcagtccctgcagacctacgtgacccagcagctgatccgcgccgccgagatccgcgcctccgccaacctggccgccaccaagatgtccgagtgcgtgctgggccagtccaagcgcgtggacttctgcggcaagggctaccacctgatgtccttcccacaggccgccccacacggcgtggtgttcctgcacgtgacctacgtgccatcccaggagcgcaacttcgccaccgccccagccatctgccacgagggcaaggcctacttcccacgcgagggcgtgttcgtgttcaacggc

SEQ ID NO: 20 - protein, 807 aa, SARS Coronavirus urbani; identical to SEQ ID NO: 4

SEQ ID NO: 21 - DNA, 744 bp, SARS Coronavirus urbani
gtgctgtacaactccgccttcttctccaccttcaagtgctacggcgtgtccgccaccaagctgaacgacctgtgcttctccaacgtgtacgccgactccttcgtggtgaagggcgacgacgtgcgccagatcgccccaggccagaccggcgtgatcgccgactacaactacaagctgccagacgacttcatgggctgcgtgctggcctggaacacccgcaacatcgacgccacctccaccggcaactacaactacaagtaccgctacctgcgccacggcaagctgcgcccattcgagcgcgacatctccaacgtgccattctccccagacggcaagccatgcaccccaccagccctgaactgctactggccactgaacgactacggcttctacaccaccaccggcatcggctaccagccataccgcgtggtggtgctgtccttcgagctgctgaacgccccagccaccgtgtgcggcccaaagctgtccaccgacctgatcaagaaccagtgcgtgaacttcaacttcaacggcctgaccggcaccggcgtgctgaccccatcctccaagcgcttccagccattccagcagttcggccgcgacgtgtccgacttcaccgactccgtgcgcgacccaaagacctccgagatcctggacatctccccatgctccttcggcggcgtgtccgtgatcaccccaggcaccaacgccgcctccgaggtggccgtgctgtaccaggacgtg

SEQ ID NO: 22 - protein, 248 aa, SARS Coronavirus urbani; identical to SEQ ID NO: 8

SEQ ID NO: 23 - DNA, 834 bp, SARS Coronavirus urbani
gccgagcaggaccgcaacacccgcgaggtgttcgcccaggtgaagcagatgtacaagaccccaaccctgaagtacttcggcggcttcaacttcgcccagatcctgccagacccactgaagccaaccaagcgctccttcatcgaggacctgctgttcaacaaggtgaccctggccgacgccggcttcatgaagcagtacggcgagtgcctgggcgacatcaacgcccgcgacctgatctgcgcccagaagttcaacggcctgaccgtgctgccaccactgctgaccgacgacatgatcgccgcctacaccgccgccctggtgtccggcaccgccaccgccggctggaccttcggcgccggcgccgccctgcagatcccattcgccatgcagatggcctaccgcttcaacggcatcggcgtgacccagaacgtgctgtacgagaaccagaagcagatcgccaaccagttcaacaaggccatctcccagatccaggagtccctgaccaccacctccaccgccctgggcaagctgcaggacgtggtgaaccagaacgcccaggccctgaacaccctggtgaagcagctgtcctccaacttcggcgccatctcctccgtgctgaacgacatcctgtcccgcctggacaaggtggaggccgaggtgcagatcgaccgcctgatcaccggccgcctgcagtccctgcagacctacgtgacccagcagctgatccgcgccgccgagatccgcgcctccgccaacctggccgccaccaagatgtccgagtgcgtgctgggccagtccaagcgcgtggacttctgcggcaagggctaccacctg

SEQ ID NO: 24 - protein, 278 aa, SARS Coronavirus urbani; identical to SEQ ID NO: 9

SEQ ID NO: 25 - DNA, 2034 bp, SARS Coronavirus urbani
gtgctgtacaactccgccttcttctccaccttcaagtgctacggcgtgtccgccaccaagctgaacgacctgtgcttctccaacgtgtacgccgactccttcgtggtgaagggcgacgacgtgcgccagatcgccccaggccagaccggcgtgatcgccgactacaactacaagctgccagacgacttcatgggctgcgtgctggcctggaacacccgcaacatcgacgccacctccaccggcaactacaactacaagtaccgctacctgcgccacggcaagctgcgcccattcgagcgcgacatctccaacgtgccattctccccagacggcaagccatgcaccccaccagccctgaactgctactggccactgaacgactacggcttctacaccaccaccggcatcggctaccagccataccgcgtggtggtgctgtccttcgagctgctgaacgccccagccaccgtgtgcggcccaaagctgtccaccgacctgatcaagaaccagtgcgtgaacttcaacttcaacggcctgaccggcaccggcgtgctgaccccatcctccaagcgcttccagccattccagcagttcggccgcgacgtgtccgacttcaccgactccgtgcgcgacccaaagacctccgagatcctggacatctccccatgctccttcggcggcgtgtccgtgatcaccccaggcaccaacgccgcctccgaggtggccgtgctgtaccaggacgtggcctgcaccgacgtgtccaccgccatccacgccgaccagctgaccccagcctggcgcatctactccaccggcaacaacgtgttccagacccaggccggctgcctgatcggcgccgagcacgtggacacctcctacgagtgcgacatcccaatcggcgccggcatctgcgcctcctaccacaccgtgtccctgctgcgctccacctcccagaagtccatcgtggcctacaccatgtccctgggcgccgactcctccatcgcctactccgccaacaccatcgccatcccaaccaacttcgccatctccatcaccaccgaggtgatgccagtgtccatggccaagacctccgtggactgcaacatgtacatctgcggcgactccaccgagtgcgccaacctgctgctgcagtacggctccttctgcacccagctgaaccgcgccctgtccggcatcgccgccgagcaggaccgcaacacccgcgaggtgttcgcccaggtgaagcagatgtacaagaccccaaccctgaagtacttcggcggcttcaacttcgcccagatcctgccagacccactgaagccaaccaagcgctccttcatcgaggacctgctgttcaacaaggtgaccctggccgacgccggcttcatgaagcagtacggcgagtgcctgggcgacatcaacgcccgcgacctgatctgcgcccagaagttcaacggcctgaccgtgctgccaccactgctgaccgacgacatgatcgccgcctacaccgccgccctggtgtccggcaccgccaccgccggctggaccttcggcgccggcgccgccctgcagatcccattcgccatgcagatggcctaccgcttcaacggcatcggcgtgacccagaacgtgctgtacgagaaccagaagcagatcgccaaccagttcaacaaggccatctcccagatccaggagtccctgaccaccacctccaccgccctgggcaagctgcaggacgtggtgaaccagaacgcccaggccctgaacaccctggtgaagcagctgtcctccaacttcggcgccatctcctccgtgctgaacgacatcctgtcccgcctggacaaggtggaggccgaggtgcagatcgaccgcctgatcaccggccgcctgcagtccctgcagacctacgtgacccagcagctgatccgcgccgccgagatccgcgcctccgccaacctggccgccaccaagatgtccgagtgcgtgctgggccagtccaagcgcgtggacttctgcggcaagggctaccacctg

SEQ ID NO: 26 - protein, 678 aa, SARS Coronavirus urbani; identical to SEQ ID NO: 10

SEQ ID NO: 27 - protein, 15 aa, SARS Coronavirus urbani
QILPDPLKPTKRSFI

SEQ ID NO: 28 - protein, 16 aa, SARS Coronavirus urbani
TRNIDATSTGNYNYKY

SEQ ID NO: 29 - protein, 5 aa, SARS Coronavirus urbani
AVDCS

SEQ ID NO: 30 - protein, 4 aa, SARS Coronavirus urbani
VFNG

SEQ ID NO: 31 - protein, 4 aa, SARS Coronavirus urbani
GYHL

SEQ ID NO: 32 - protein, 4 aa, SARS Coronavirus urbani
YQDV

SEQ ID NO: 33 - protein, 6 aa, SARS Coronavirus urbani
AEQDRN